(19) United States
(12) Patent Application Publication
(10) Pub. No.: US 2015/0320024 A1
(43) Pub. Date: Nov. 12, 2015

(54) BIODEGRADABLE WORM-EGG-DELIVERY SYSTEM FOR SOIL ENHANCEMENT AND METHODS OF USE

(71) Applicant: Ingenious Marketing LLC, Denver, CO (US)

(72) Inventor: Gail D. LOOS, Centennial, CO (US)

(73) Assignee: INGENIOUS MARKETING LLC, Denver, CO (US)

(21) Appl. No.: 14/700,115

(22) Filed: Apr. 29, 2015

Related U.S. Application Data
(60) Provisional application No. 61/989,529, filed on May 7, 2014.

(51) Int. Cl. A01K 67/033; B65B 25/02; B65B 5/06; B65D 65/46; B65D 75/00

(52) U.S. Cl. CPC A01K 67/0332; B65D 65/466; B65D 75/002; B65B 5/06; B65B 25/02; B65D 2565/382

(57) ABSTRACT

The present inventive disclosure is generally directed to a biodegradable device that can house multiple worm-cocoon capsules, as well as an effective amount of shredded organic fibrous materials (such as coir fiber or shredded paper) that is infused with hormones, nutrients, and/or amino acids that are beneficial to earthworms. In some embodiments, these devices are referred to as “Worm Bombs™”, which are planted at effective intervals in gardens and/or compost heaps, leading to stable and vigorous earthworm populations and softened, nutrient-rich soil that is ideal for growing. It is known in the art that earthworm biomass production can be significantly enhanced with the introduction of growth hormones, which inter alia, increases the efficiency of waste conversion of worms.

### CROSS-REFERENCE TO RELATED PATENT APPLICATIONS

The present patent application claims the priority benefit of United States Provisional Patent Application No. 61/989,529 for “Biodegradable Worm-Egg Delivery System and Method of Use”, filed on May 7, 2014. Moreover, U.S. Provisional Patent Application No. 61/989,529 is hereby incorporated by reference herein for all purposes.

### BACKGROUND

It is well-known that gardens generally perform better when their soils are loose, allowing for better aeration and hydration. Toward this end, nature provides a “plow” of sorts in the form of earthworms. Earthworms eat their way through hard-packed soil, forming interconnected burrows that can become several feet deep. Such burrows loosen the soil, which in turn admits air and water leading to healthier plant roots in gardens.

Earthworms can thrive in gardens that are naturally fertilized and nitrogen-rich; however, earthworms are sensitive to physical and chemical changes and will flee the salty conditions that result from an application of artificial chemical fertilizers. As an earthworm feeds, organic matter passes through its body and is excreted as nutrient-rich, granular, dark castings. An earthworm produces up to its weight in castings daily. Earthworm castings make wonderful fertilizer, rich in nutrients otherwise unavailable to plants.

In cold weather, a soil search will often turn up mature and young earthworms as well as eggs. By late spring, most earthworms are mature. As temperatures rise, activity slows; many lay eggs and then die. By midsummer, most earthworms are very young or protected by egg capsules. As the weather cools, young earthworms emerge. With wet weather, they grow active, making new burrows and eating extra food, resulting in more worm castings, and egg laying occurs again. This activity continues as long as soil stays damp. Earthworms tend to be hardy as long as they have access to moisture, but can be killed; for example, during sudden freezes.

Many gardeners try to increase the population of earthworms in their gardens and other horticultural endeavors by creating earthworm bins for raising populations of earthworms; that is, compost heaps or vermiculture. Such earthworm compost heaps generally must be kept in a cool, dark place, such as a basement. This earthworm compost heap is periodically mined to transfer compost and earthworms to a user's garden to keep the garden healthy and thriving.

Earthworm compost bins are typically made from durable plastic or wood, with trays that fit on top of a base. Bins are often layered in organic materials and soil, with the lowest layer having moist filler material (usually soaked coir fiber brick and/or shredded paper, with a little compost or garden soil mixed in to provide beneficial organisms), and are kept moist, but well-drained. Earthworms are added, and hopefully thrive and multiply as kitchen scraps and other beneficial organics are added to the compost pile. Periodically, some or all of the earthworm-processed compost is harvested for use in a garden; however, if the earthworm population is desired to be used for another round of compost processing, the earthworms must be segregated from the compost before spreading in a garden, which is messy and labor-intensive. In addition, it would be desirable to have a convenient and ready supply of earthworms to introduce to both the compost heap and the garden.

Vermipods™ are products on the market that are comprised of one or more earthworm eggs encapsulated by a layer of protective clay (although in some cases, a vermipod may only be a worm cocoons without any added protective clay layer), sometimes with some additional nutrients added. There are other similar worm-cocoon capsules on the market as well, though Vermipods™ are probably best known. However, by themselves, once hatched, the earthworms still need a nutrient-rich and moist environment to thrive and grow. Further, it would be desirable to create an environment such that earthworms will not be inclined to migrate away from the target soil that is being cultivated. What is needed is a convenient delivery system that is ecologically friendly, yet convenient and relatively maintenance-free to introduce new earthworms to soils and compost heaps, as well as to encourage newly hatched earthworms to remain in the target area to grow and thrive.

### BRIEF SUMMARY

The present inventive disclosure is generally directed to a biodegradable capsule/shell that can house multiple worm-cocoon capsules, as well as an effective amount of organic fibrous materials (such as coir fiber or shredded corrugated cardboard) that is infused with growth hormones, essential amino acids, and/or nutrients beneficial to earthworms. In some embodiments, these worm-egg-delivery devices are referred to herein as “Worm Bombs™”, which are planted at effective intervals in gardens and/or compost heaps, leading to increased earthworm populations and softened, nutrient-rich soil that is ideal for growing. It is known in the art that earthworm biomass production can be significantly enhanced with the introduction of growth hormones, which inter alia, increases the efficiency of waste conversion of worms. However, the presently claimed invention introduces the novel combination of the aforementioned components in a capsule/shell to create a highly effective delivery system for worm eggs that promotes the hatching and growth and health of the worms long after planting the Worm Bomb™. In addition, in some variations, a Worm Bomb™ incorporates one or more essential amino acids, such as tryptophan and leucine, which encourages composting worms to stay and dwell versus roam a yard and helps build muscle tissue in the earthworms.

The foregoing Brief Summary is intended to merely provide a short, general overview of the inventive disclosure described throughout this document, and therefore, is not intended to limit the scope of the inventive disclosure contained throughout the balance of this document and its appended drawings.

### BRIEF DESCRIPTION OF THE DRAWINGS

FIGS. 1A and 1B each depict one embodiment of a worm-egg-delivery device's internal and external components, respectively.

### DETAILED DESCRIPTION

### I. Terminology

The terms and phrases as indicated in quotes (“ ”) in this Section are intended to have the meaning ascribed to them in this Terminology Section applied to them throughout this document, including the claims, unless clearly indicated otherwise in context. Further, as applicable, the stated definitions are to apply, regardless of the word or phrase's case, to the singular and plural variations of the defined word or phrase.

The term “or”, as used in this specification, drawings, and any appended claims, is not meant to be exclusive; rather, the term is inclusive, meaning “either or both”.

References in the specification to “one embodiment”, “an embodiment”, “a preferred embodiment”, “an alternative embodiment”, “a variation”, “one variation”, and similar phrases mean that a particular feature, structure, or characteristic described in connection with the embodiment is included in at least an embodiment of the invention. The appearances of the phrase “in one embodiment” and/or “in one variation” and similar phrases in various places in the specification are not necessarily all meant to refer to the same embodiment.

The term “couple” or “coupled”, as used in this specification, drawings, and any appended claims, refers to either an indirect or a direct connection between the identified elements, components, or objects. Often the manner of the coupling will be related specifically to the manner in which the two coupled elements interact.

The term “removable”, “removably coupled”, “readily removable”, “readily detachable”, “detachably coupled”, and similar terms, as used in this specification, drawings, and any appended claims, refer to structures that can be uncoupled from an adjoining structure with relative ease (i.e., non-destructively and without a complicated or time-consuming process) and that can also be readily reattached or coupled to the previously adjoining structure.

Directional and/or relational terms such as, but not limited to, left, right, nadir, apex, top, bottom, vertical, horizontal, back, front, lateral, proximal, and distal are relative to each other, are dependent on the specific orientation of an applicable element or article, are used accordingly to aid in the description of the various embodiments, and are not necessarily intended to be construed as limiting in this specification, drawings, and any appended claims.

As applicable, the terms “about” or “generally”, as used herein unless otherwise indicated, means a margin of +−20%. Also, as applicable, the term “substantially” as used herein unless otherwise indicated means a margin of +−10%. It is to be appreciated that not all uses of the above terms are quantifiable such that the referenced ranges can be applied.

The term “worm-cocoon capsule” and “Vermipod™”, as used in this specification, drawings, and any appended claims, refers to any discrete capsule-like units comprised of one or more worm cocoons, wherein each worm cocoon can house one or more worm eggs. Worms hatch from a worm cocoon. A worm-cocoon capsule may be further encapsulated by a mineral-based protective layer (e.g., a layer of protective clay, as in the case of “Vermipods™”) or by a plant-based layer (e.g., a protective layer based on alginate). In addition, in some cases, a worm-cocoon capsule may only consist of one or more worm cocoons without any added protective layer. In some cases, worm-cocoon capsules also include some added nutrients.

### II. Overview of the Inventive Disclosure

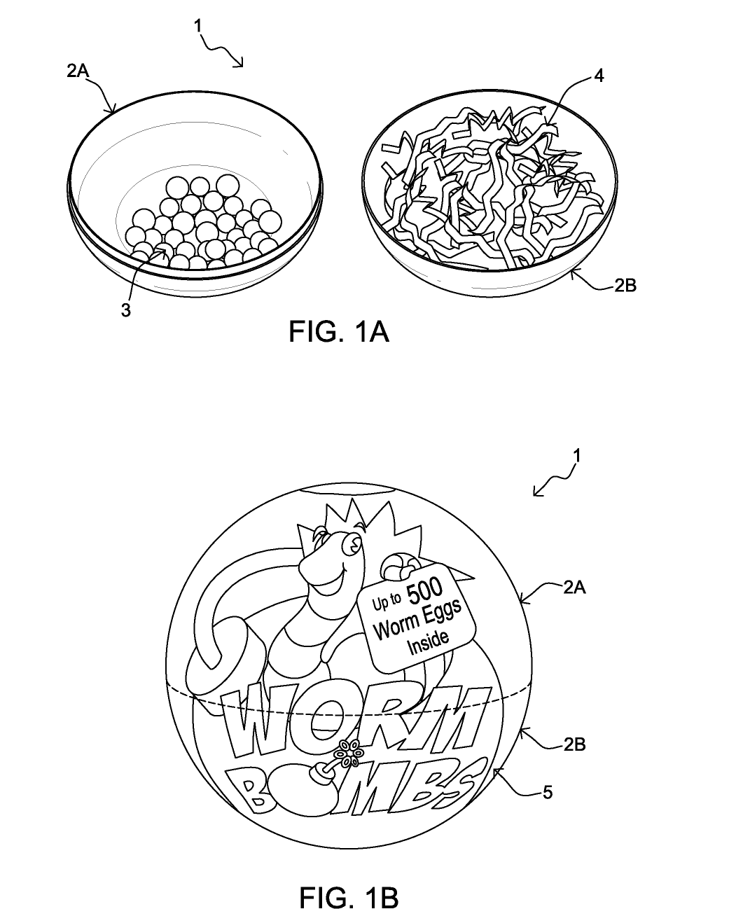

Refer to FIGS. 1A-1B. The present inventive disclosure is generally directed to a novel combination of a biodegradable capsule or shell 2A, 2B that can house multiple worm-cocoon capsules 3, as well as an effective amount of shredded organic fibrous materials 4 (such as, for example, coir fiber or shredded worm-growth paper) that is infused with hormones and/or other nutrients and substances beneficial to earthworms. In many embodiments, The biodegradable capsule/shell 2A, 2B is comprised of two mated hollow shells 2A, 2B that contain one or more worm-cocoon capsules 3, as well as an effective amount of shredded organic fibrous materials 4 to serve as food for newly hatched worms. In variations, covering the entire assembled housing is a biodegradable shrink wrap 5 that often will have packaging labeling on it. In many embodiments, these assembled worm-egg-delivery devices 1 are referred to as “Worm Bombs™” 1.

It is known in the art that earthworm biomass production can be significantly enhanced with the introduction of growth hormones, which inter alia, increases the efficiency of waste conversion of worms. See, e.g., Patrabansh, S., “Can Hormones Increase Earthworm Biomass Production”, Article, Biocycle, Dec. 2004, Vol. 45, Issue 12, p 49, which found that earthworm biomass was optimized by using gibberellic acid (hormone GA) at a concentration of around 50-4000 ppm (but preferably 2000 ppm), and similarly in the use of naphthaleneacetic acid (hormone NAA) also at a concentration at around 50-4000 ppm (but preferably 2000 ppm). However, the issue becomes how to effectively and efficiently do so, especially for individual gardeners and small growers. Accordingly, in an embodiment, the shredded organic fibrous materials 4 are infused and/or coated with ligand molecules or similar worm-growth stimulant that is known in the art.

However, in other embodiments, the shredded organic fibrous materials 4 are infused with, and/or mixed with, substances/compounds rich in certain amino acids instead of or in addition to other helpful compounds discussed herein. For example, tryptophan is one of the 22 standard amino acids and an essential amino acid in the human diet, and is commonly referred to as the “feel good” amino acid that in humans is believed to contribute to feelings of well being, relaxation, and social interaction and mood. In embodiments of the present inventive disclosure, tryptophan-rich foods can help composting worms be more-resistant to stresses from temperature fluctuations, carbon or nitrogen shortages, adverse soil conditions, etc. Tryptophan is a precursor to Serotonin which, in humans, has positive effects related to sleep, depression, anxiety, aggression, appetite, temperature, sexual behavior, and pain sensation. Moreover, researchers at a number of research universities, including Harvard University, have determined that serotonin causes “dwelling” behavior in caenorhabditis elegans (aka “C. elegans”), which are free-living unsegmented pseudocoelomate nematodes that live in temperate soil environments. See, e.g., Flavell, Steven W. et al., “Serotonin and the Neuropeptide PDF Initiate and Extend Opposing Behavioral States in C. elegans”, Cell, Issue 5 , 1023-1035, 2013 Aug. 29, Volume 154; and Srinivasan, Supriya et al., “Seratonin Regulates C. elegans Fat and Feeding through Independent Molecular Mechanisms”; Cell Metabolism 7, 533-544, Jun. 2008; the teachings of which are hereby incorporated by reference. What was not apparent from those studies is whether those teaching could be applied to more-complex organisms, such as earthworms. Accordingly, some embodiments of the present inventive disclosure leverage the teachings of the aforementioned research by applying them to earthworms; that is, exposing earthworms and their offspring to serotonin-enhancing food to cause composting earthworms to stay and dwell versus roam the yard. Dwelling in a localized area translates into more feeding and mating; hence, an increased and stable earthworm population in the localized area.

Another amino acid that can be used in some variations is leucine, which is a branched-chain α-amino acid, and is also considered an essential amino acid for humans, meaning that the human body cannot synthesize it and so it must be ingested. Leucine is associated with helping the human body detect its protein levels, and is central to muscle-building in humans. Since composting earthworms are mainly muscle, it is believed that leucine can help earthworms increase overall their strength, size, and vigor.

Leucine and tryptophan compete for uptake in humans. Further, these essential amino acids are only available through dietary sources. Therefore, foods that are high in both leucine and tryptophan would theoretically provide balance between these two amino acids. Abstracting this concept further, if earthworms were exposed to composting foods high in leucine and tryptophan, then theoretically, the earthworms would remain localized while continuing to boost a healthy earthworm population by feeding and mating.

Corrugated cardboard (and the animal-hide glue that holds it together) is used as another favorite food/bedding material for composting worms. Therefore, in an embodiment, the shredded organic fibrous materials 4 are comprised of shredded corrugated cardboard combined with the powdered form of leucine and tryptophan and/or combined with the dried natural form of pumpkin, squash, and/or similar members of the gourd family. Further, in variations, the shredded organic fibrous materials 4 are comprised of pelletized compost containing significant quantities of pumpkin, squash, and/or similar members of the gourd family.

In an embodiment, organic fibrous materials 4 are introduced to worm stimulant or growth accelerants, and/or hormones, and/or the aforementioned essential amino acids during the pulping process as recycled paper and/or cardboard is being recovered. However, in other embodiments, these same worm stimulant or growth accelerants, hormones, and/or the aforementioned essential amino acids, in differing volumes, can be added as a powder during the shredding of original wood material and/or added as a liquified wash during the final step of finishing the paper. In general, dried additives tend to be more concentrated and, therefore, can be added at much-lower volumes. In some embodiments, the amount of powdered or re-hydrated worm stimulant or growth accelerants, and/or hormones, and/or the aforementioned essential amino acids added to any material should not exceed 0.05% of volume by weight.

Alternatively in some embodiments, the shredded organic fibrous materials 4 do not have any additional worm-growth stimulant added.

In an embodiment, the biodegradable capsule/shell 2A, 2B is comprised of a biodegradable/compostable material, such as agro-polymers. In preferred variations, biodegradable/compostable material is comprised of a polymer made from potato starch, corn starch, sugar beet starch, or similar natural starch, which is also edible by worm hatchlings. Similarly, in embodiments, the shrink wrap 5 covering most of the capsule/shell 2A, 2B is also made of a biodegradable material such as a polymer substantially made from a vegetable-based starch.

In other embodiments, the biodegradable capsule/shell 2A, 2B is comprised of a pressed and dried composite material that includes materials selected from the group consisting of compost, wood shavings, soil, clay, and a combination of any of the aforementioned materials, and the like to form a more-readily breachable and decomposable capsule/shell 2A/2B to facilitate the effective planting of planting of discrete Worm Bombs™ 1. In variations, these pressed shell components can include soil and other mineral-based components.

In some embodiments, the capsule/shell 2A, 2B is spherical in shape (however, other shapes are possible and contemplated), and has two hemispherical halves 2A, 2B that can be detachably coupled to each other. Helping to hold the shell members 2A, 2B together in some variations is a biodegradable/compostable shrink-wrapped product sleeve 5 substantially comprised of an agro-polymer, such as, but not limited to, a natural starch polymer (such as a vegetable-based-starch polymer), as previously described, supra. This sleeve 5 is similarly edible to worm hatchlings.

While hard, the capsule/shell 2A, 2B is typically thin enough for a user to easily be able to crack it/breach the enclosure 2A, 2B, which is desirable just before burial to help prompt the hatching and enable the migration of new earthworms as moisture enters the enclosure 2A, 2B via the breach.

In one method of use, after breaching or creating an opening in a capsule/shell 2A, 2B, the capsule/shell 2A, 2B is simply buried in soil or compost such that the contained earthworm cocoons/eggs can hatch in an ideal environment with minimal or no maintenance. This system 1 allows the efficient and effective delivery of literally hundreds of earthworms into an ecosystem per capsule/shell 2A, 2B. In many embodiments, the capsule/shell 2A, 2B is generally small enough to easily hold in a user hand (typically, 3-12 inches in diameter), and in one preferred variation contains 50 worm-cocoon capsules 3, with each worm-cocoon capsule 3 holding 10 earthworm cocoons, each containing at least one worm egg (that is, each Worm Bomb™ can yield 500 or more earthworms).

This system 1 can facilitate the delivery of any number of species of earthworms in various versions of the worm-egg-delivery device 1; for example, red wigglers, tiger worms or other species specific to a particular environment or purpose. In many embodiments, either a single species or a combination of as many as six or more species are used to maximize the adaptability of hatchlings to different soil conditions, humidity levels, climates, and feed stocks.

Of course, those skilled in the art would appreciate that other numbers of worm-cocoon capsules 3, comprised of other numbers of eggs per worm-cocoon capsule 3, and comprised of multiple species of worms eggs in the same capsule/shell 2A, 2B are possible.

Worm Bombs™ 1 can be planted in gardens like seeds, and in one embodiment of such uses, are planted at a density of one Worm Bomb 1 per 50 square feet, with the earthworms hatching in 10 to 22 days, depending on environmental conditions, species, etc. In many embodiments, this planting density will translate into an average of one worm-cocoon capsule per square foot.

### III. A Worm-Egg-Delivery Device

Refer to FIGS. 1A-1B. This section is generally directed to a worm-egg-delivery device comprised of: a biodegradable container shell 2A, 2B, having a first shell member 2A and a second shell member 2B, wherein the first and second shell members 2A, 2B are adapted to be coupled to one another and the container shell 2A, 2B has an internal volume containing at least one worm-cocoon capsule 3 and organic fibrous material 4.

In an embodiment, the biodegradable container shell 2A, 2B is substantially comprised of an agro-polymer, such as, but not limited to, a natural starch polymer. In variations, the biodegradable container shell 2A, 2B is substantially comprised of a polymeric material selected from the group consisting of potato starch, corn starch, rice starch, and sugar-beet starch.

In other embodiments, the biodegradable container shell 2A, 2B is comprised of a pressed and dried composite material that includes materials selected from the group consisting of compost, wood shavings, soil, clay, and a combination of any of the aforementioned materials, and the like to form a more-readily breachable and decomposable container shell 2A/2B to facilitate the effective planting of planting of discrete Worm Bombs™ 1. In variations, these pressed shell components can include soil and other mineral-based components.

In an embodiment, the worm-egg-delivery device further comprises a biodegradable shrink-wrap sleeve 5 to help hold the shell members 2A, 2B together. In variations, the biodegradable shrink-wrap sleeve 5 is substantially comprised of an agro-polymer, such as, but not limited to, a natural starch polymer. In more variations, the biodegradable shrink-wrap sleeve 5 is substantially comprised of a polymeric material selected from the group consisting of potato starch, corn starch, rice starch, and sugar-beet starch.

In an embodiment, the organic fibrous material 4 is substantially comprised of one or more materials selected from the group consisting of shredded corrugated cardboard, shredded paper, pelletized compost, coir fiber, peat moss, and shredded worm-growth paper. In variations, the organic fibrous material 4 is infused and/or coated with a material containing amino acids selected from the group consisting of leucine, tryptophan, or both leucine and tryptophan. In other variations, the amino acids are provided via a material selected from the group consisting of a powdered form of leucine, a powdered form of tryptophan, a powdered form of leucine and tryptophan, a dried natural form of pumpkin, a dried natural form of squash, a dried natural form of members of the gourd family, and a combination of any of the aforementioned materials.

In an embodiment, organic fibrous materials 4 are introduced to worm stimulant or growth accelerants, and/or hormones, and/or the aforementioned essential amino acids during the pulping process as recycled paper and/or cardboard is being recovered. However, in other embodiments, these same worm stimulant or growth accelerants, hormones, and/or the aforementioned essential amino acids, in differing volumes, can be added as a powder during the shredding of original wood material and/or added as a liquified wash during the final step of finishing the paper. In general, dried additives tend to be more concentrated and, therefore, can be added at much-lower volumes. In some embodiments, the amount of powdered or re-hydrated worm stimulant or growth accelerants, and/or hormones, and/or the aforementioned essential amino acids added to any material should not exceed 0.05% of volume by weight.

In an embodiment, the plurality of worm-cocoon capsules 3 includes worm eggs for species selected from the group consisting of red wigglers, tiger worms, eisenia fetida, lumbricus terrestris Linnaeus, perionyx excavatus Perrier, lampito mauritii Kinberg, criodrilus lacuum Hoffmeister, and the common nightcrawler.

### IV. A Method of Making a Worm-Egg-Delivery Device

Refer to FIGS. 1A-1B. This section is generally directed to a method of making a worm-egg-delivery device comprising the steps of: providing a biodegradable container shell 2A, 2B, having a first shell member 2A and a second shell member 2B, wherein the first and second shell members 2A, 2B are adapted to be coupled to one another and the container shell 2A, 2B has an internal volume; providing at least one worm-cocoon capsule 3; and providing organic fibrous material 4.

In an embodiment, the biodegradable container shell 2A, 2B is substantially comprised of an agro-polymer, such as, but not limited to, a natural starch polymer. In variations, the biodegradable container shell 2A, 2B is substantially comprised of a polymeric material selected from the group consisting of potato starch, corn starch, rice starch, and sugar-beet starch.

In other embodiments, the biodegradable container shell 2A, 2B is comprised of a pressed and dried composite material that includes materials selected from the group consisting of compost, wood shavings, soil, clay, and a combination of any of the aforementioned materials, and the like to form a more-readily breachable and decomposable container shell 2A/2B to facilitate the effective planting of planting of discrete Worm Bombs™ 1. In variations, these pressed shell components can include soil and other mineral-based components.

In an embodiment, the method further comprises the step of providing a biodegradable shrink-wrap sleeve 5 to help hold the shell members 2A, 2B together. In variations, the biodegradable shrink-wrap sleeve 5 is substantially comprised of an agro-polymer, such as, but not limited to, a natural starch polymer. In more variations, the biodegradable shrink-wrap sleeve 5 is substantially comprised of a polymeric material selected from the group consisting of potato starch, corn starch, rice starch, and sugar-beet starch.

In an embodiment, the organic fibrous material 4 is substantially comprised of one or more materials selected from the group consisting of shredded corrugated cardboard, shredded paper, pelletized compost, coir fiber, peat moss, and shredded worm-growth paper. In variations, the method further comprises the step of infusing and/or coating the organic fibrous material 4 with a material containing amino acids selected from the group consisting of leucine, tryptophan, or both leucine and tryptophan. In other variations, the amino acids are provided via a material selected from the group consisting of a powdered form of leucine, a powdered form of tryptophan, a powdered form of leucine and tryptophan, a dried natural form of pumpkin, a dried natural form of squash, a dried natural form of members of the gourd family, and a combination of any of the aforementioned materials.

In an embodiment, organic fibrous materials 4 are introduced to worm stimulant or growth accelerants, and/or hormones, and/or the aforementioned essential amino acids during the pulping process as recycled paper and/or cardboard is being recovered. However, in other embodiments, these same worm stimulant or growth accelerants, hormones, and/or the aforementioned essential amino acids, in differing volumes, can be added as a powder during the shredding of original wood material and/or added as a liquified wash during the final step of finishing the paper. In general, dried additives tend to be more concentrated and, therefore, can be added at much-lower volumes. In some embodiments, the amount of powdered or re-hydrated worm stimulant or growth accelerants, and/or hormones, and/or the aforementioned essential amino acids added to any material should not exceed 0.05% of volume by weight.

In an embodiment, the plurality of worm-cocoon capsules 3 includes worm eggs for species selected from the group consisting of red wigglers, tiger worms, eisenia fetida, lumbricus terrestris Linnaeus, perionyx excavatus Perrier, lampito mauritii Kinberg, criodrilus lacuum Hoffmeister, and the common nightcrawler.

### V. A Method of Using a Worm-Egg-Delivery Device

Refer to FIGS. 1A-1B. This section is generally directed to a method of using a worm-egg-delivery device according to Sections II or III, supra, comprising the steps of: obtaining at least one worm-egg-delivery device 1 according to Sections II or III, supra; cracking or otherwise breaching the outer container shell 2A, 2B of the at least one worm-egg-delivery device 1; and planting said worm-egg-delivery device 1 in user-selected cultivated soil. In a variation, the planting density is one Womb Bomb™ 1 per 50 square feet of garden surface area. In many embodiments, this planting density will translate into an average of one worm-cocoon capsule per square foot.

In variations, the method can be extended by further comprising the step of selecting at least one worm-egg-delivery device 1 that is configured to deliver worm eggs for species selected from the group consisting of red wigglers, tiger worms, eisenia fetida, lumbricus terrestris Linnaeus, perionyx excavatus Perrier, lampito mauritii Kinberg, criodrilus lacuum Hoffmeister, and the common nightcrawler. In still more variations, at least one worm-egg-delivery device contains eggs for multiple worm species.

### VI. Alternative Embodiments and Other Variations

The various embodiments and variations thereof described herein, including the descriptions in any appended Claims and/or illustrated in the accompanying Figures, are merely exemplary and are not meant to limit the scope of the inventive disclosure. It should be appreciated that numerous variations of the invention have been contemplated as would be obvious to one of ordinary skill in the art with the benefit of this disclosure.

Hence, those ordinarily skilled in the art will have no difficulty devising myriad obvious variations and improvements to the invention, all of which are intended to be encompassed within the scope of the Description, Claims, and Figures herein.

What is claimed is:

1. A worm-egg-delivery device:
a biodegradable container shell, having a first shell member and a second shell member, wherein:
said first and second shell members are adapted to be coupled to one another, and
said container shell has an internal volume;
at least one worm-cocoon capsule; and
organic fibrous material.

2. The worm-egg-delivery device of claim 1, wherein said biodegradable container shell is substantially comprised of an agro-polymer.

3. The worm-egg-delivery device of claim 2, wherein said biodegradable container shell is substantially comprised of a polymeric material selected from the group consisting of potato starch, corn starch, rice starch, and sugar-beet starch.

4. The worm-egg-delivery device of claim 1, wherein said biodegradable container shell is comprised of a pressed and dried composite material that includes materials selected from the group consisting of compost, wood shavings, soil, clay, and a combination of any of the aforementioned materials.

5. The worm-egg-delivery device of claim 1, further comprising a biodegradable shrink-wrap sleeve to help hold said shell members together.

6. The worm-egg-delivery device of claim 5, wherein said biodegradable shrink-wrap sleeve is substantially comprised of an agro-polymer.

7. The worm-egg-delivery device of claim 5, wherein said biodegradable shrink-wrap sleeve is substantially comprised of a polymeric material selected from the group consisting of potato starch, corn starch, rice starch, and sugar-beet starch.

8. The worm-egg-delivery device of claim 1, wherein said organic fibrous material is substantially comprised of one or more materials selected from the group consisting of shredded corrugated cardboard, shredded paper, pelletized compost, coir fiber, peat moss, and shredded worm-growth paper.

9. The worm-egg-delivery device of claim 1, wherein said organic fibrous material is infused and/or coated with a material containing amino acids selected from the group consisting of leucine, tryptophan, or both leucine and tryptophan.

10. The worm-egg-delivery device of claim 9, wherein said amino acids are provided via a material selected from the group consisting of a powdered form of leucine, a powdered form of tryptophan, a powdered form of leucine and tryptophan, a dried natural form of pumpkin, a dried natural form of squash, a dried natural form of members of the gourd family, and a combination of any of the aforementioned materials.

11. The worm-egg-delivery device of claim 1, wherein said organic fibrous material is infused and/or coated with 50 to 4000 ppm of a hormone selected from the group consisting of GA hormone and NAA hormone.

12. The worm-egg-delivery device of claim 1, wherein said plurality of worm-cocoon capsules includes worm eggs for species selected from the group consisting of red wigglers, tiger worms, eisenia fetida, lumbricus terrestris Linnaeus, perionyx excavatus Perrier, lampito mauritii Kinberg, criodrilus lacuum Hoffmeister, and the common nightcrawler.

13. A method of making a worm-egg-delivery device, comprising the steps of:
providing a biodegradable container shell, having a first shell member and a second shell member, wherein:
said first and second shell members are adapted to be coupled to one another, and
said container shell has an internal volume;
providing at least one worm-cocoon capsule;
providing organic fibrous material; and
infusing and/or coating said organic fibrous material with a material containing amino acids selected from the group consisting of leucine, tryptophan, or both leucine and tryptophan.

14. The method of claim 13, wherein said biodegradable container shell is substantially comprised of an agro-polymer or of a pressed and dried composite material that includes materials selected from the group consisting of compost, wood shavings, soil, clay, and a combination of any of the aforementioned materials.

15. The method of claim 13, further comprising the step of providing a biodegradable shrink-wrap sleeve to help hold said shell members together, wherein said biodegradable shrink-wrap sleeve is substantially comprised of an agro-polymer.

16. The method of claim 13, wherein said organic fibrous material is substantially comprised of one or more materials selected from the group consisting of shredded corrugated cardboard, shredded paper, pelletized compost, coir fiber, peat moss, and shredded worm-growth paper.

17. The method of claim 13, wherein said amino acids are provided via a material selected from the group consisting of a powdered form of leucine, a powdered form of tryptophan, a powdered form of leucine and tryptophan, a dried natural form of pumpkin, a dried natural form of squash, a dried natural form of members of the gourd family, and a combination of any of the aforementioned materials.

18. The method of claim 13, wherein said organic fibrous material is infused and/or coated with 50 to 4000 ppm of a hormone selected from the group consisting of GA hormone and NAA hormone.

19. The method of claim 13, wherein said plurality of worm-cocoon capsules includes worm eggs for species selected from the group consisting of red wigglers, tiger worms, eisenia fetida, lumbricus terrestris Linnaeus, perionyx excavatus Perrier, lampito mauritii Kinberg, criodrilus lacuum Hoffmeister, and the common nightcrawler.

20. A method of using a worm-egg-delivery device according to claim 1, comprising the steps of:
obtaining at least one worm-egg-delivery device according to claim 1,
wherein said biodegradable container shell for said at least one worm-egg-delivery device is substantially comprised of an agro-polymer or of a pressed and dried composite material that includes materials selected from the group consisting of compost, wood shavings, soil, clay, and a combination of any of the aforementioned materials;
cracking or otherwise breaching the outer container shell of said at least one worm-egg-delivery device; and
planting said at least one worm-egg-delivery device in user-selected cultivated soil.

21. The method of claim 20, wherein said at least one worm-egg-delivery device further comprises a biodegradable shrink-wrap sleeve to help hold said shell members together, wherein said biodegradable shrink-wrap sleeve for said at least one worm-egg-delivery device is substantially comprised of an agro-polymer.

22. The method of claim 20, wherein:
said organic fibrous material for said at least one worm-egg-delivery device is substantially comprised of one or more materials selected from the group consisting of shredded corrugated cardboard, shredded paper, pelletized compost, coir fiber, and shredded worm-growth paper;
said at least one worm-egg-delivery device further comprises said organic fibrous material being infused and/or coated with a material containing amino acids selected from the group consisting of leucine, tryptophan, or both leucine and tryptophan; and
said amino acids are provided via a material selected from the group consisting of a powdered form of leucine, a powdered form of tryptophan, a powdered form of leucine and tryptophan, a dried natural form of pumpkin, a dried natural form of squash, a dried natural form of members of the gourd family, and a combination of any of the aforementioned materials.

23. The method of claim 20, wherein:
said organic fibrous material for said at least one worm-egg-delivery device is substantially comprised of one or more materials selected from the group consisting of shredded corrugated cardboard, shredded paper, pelletized compost, coir fiber, and shredded worm-growth paper;
said at least one worm-egg-delivery device further comprises said organic fibrous material being infused and/or coated with a material containing infused and/or coated with 50 to 4000 ppm of a hormone selected from the group consisting of GA hormone and NAA hormone.

24. The method of claim 20, wherein said plurality of worm-cocoon capsules for said at least one worm-egg-delivery device includes worm eggs for species selected from the group consisting of red wigglers, tiger worms, eisenia fetida, lumbricus terrestris Linnaeus, perionyx excavatus Perrier, lampito mauritii Kinberg, criodrilus lacuum Hoffmeister, and the common nightcrawler.
